(19) United States
(12) Patent Application Publication
(10) Pub. No.: US 2017/0319653 A1
(43) Pub. Date: Nov. 9, 2017

(54) METHOD OF TREATING MUCUS HYPERSECRETION

(71) Applicant: PARANTA BIOSCIENCES LIMITED, Southbank (AU)

(72) Inventors: Robyn O'Hehir, Parkville (AU); Charles Hardy, Brunswick (AU); David De Kretser, Surrey Hills (AU)

(73) Assignee: PARANTA BIOSCIENCES LIMITED, Southbank (AU)

(21) Appl. No.: 15/603,967

(22) Filed: May 24, 2017

Related U.S. Application Data
(63) Continuation of application No. 14/354,403, filed on Apr. 25, 2014, now abandoned.

(30) Foreign Application Priority Data
Oct. 28, 2011 (AU) 2011904500

(51) Int. Cl. A61K 38/17; A61K 31/7105; A61K 31/711

(52) U.S. Cl. CPC A61K 38/1709; A61K 38/1703; A61K 31/7105; A61K 31/711

(57) ABSTRACT

The present invention relates generally to a method of reducing unwanted airway tissue mucus secretion in a mammal and to agents useful for same. More particularly, the present invention relates to a method of reducing airway tissue mucus hypersecretion in a mammal by downregulating the functional level of activin or upregulating the functional level of follistatin. The method of the present invention is useful, inter alia, in the treatment and/or prophylaxis of conditions characterised by airway tissue mucus dysfunction, such as overproduction of mucus or decreased mucus clearance, and where a reduction in mucus secretion levels would thereby alleviate the condition.

### FIELD OF THE INVENTION

The present invention relates generally to a method of reducing unwanted airway tissue mucus secretion in a mammal and to agents useful for same. More particularly, the present invention relates to a method of reducing airway tissue mucus hypersecretion in a mammal by downregulating the functional level of activin or upregulating the functional level of follistatin. The method of the present invention is useful, inter alia, in the treatment and/or prophylaxis of conditions characterised by airway tissue mucus dysfunction, such as overproduction of mucus or decreased mucus clearance, and where a reduction in mucus secretion levels would thereby alleviate the condition.

### BACKGROUND OF THE INVENTION

Bibliographic details of the publications referred to by author in this specification are collected alphabetically at the end of the description.

The reference in this specification to any prior publication (or information derived from it), or to any matter which is known, is not, and should not be taken as an acknowledgment or admission or any form of suggestion that prior publication (or information derived from it) or known matter forms part of the common general knowledge in the field of endeavour to which this specification relates.

Mucus secretion in the airways normally represents the first-line defence of the respiratory tract and is an important feature of the innate immune system. It is for this reason that the lungs are so resistant to environmental injury, despite continuous exposure to pathogens, particles and toxins in the inhaled air. The mucus which protects the airway surface from these antigens is a complex non-homogenous dilute (1-2%) aqueous solution of electrolytes, endogenous and exogenous proteins, lipids and carbohydrates. Mucus forms an upper gel layer and a lower sol layer.

Mucus contains ˜2% mucins (Davies et al. 2002, Novartis Foundation Symposium 248. pp. 76-93), which are high molecular weight glycoproteins that confer the viscoelasticity required for efficient mucus-cilia interaction. Airway mucins are secreted by goblet cells in the surface epithelial (Rogers 2003, Int. J. Biochem. Cell Biol. 35:1-6) and mucous cells in the submucosal glands (Finkbeiner 1999, Respir Physiol. 118:77-83). Mature mucins are long thread-like molecules composed of monomers joined end to end by disulphide bridges. Unlike the mucus layers of the gut, which are thick, the mucus layers of the airway are thin and mobile. Accordingly, this facilitates the trapping of inhaled particles by the mucus and, by transportation on the tips of beating cilia, removal from the airways. This process is termed mucociliary clearance.

The secretion of polymeric mucins is regulated separately from mucin production (Davis and Dickey 2008, Annu Rev Physiol 70:487-512; Adler and Li 2001, Am. J. Respir. Cell Molec. Biol. 25:397-400). The most important secretagogue for surface epithelium appears to be ATP, which acts on apical membrane P2Y₂ receptors (Kim et al. 2003, J. Phoarmacol. Sci, 92:301-307; Lazarowski and Boucher 2009, Curr Opin Pharmacol. 9:262-267; Davis and Lazarowski 2008, Respir. Physiol. Neurobiol. 163:208-213). The continuous presence of low levels of ATP in airway-surface liquid causes continuous low activity of the secretory machinery, resulting in the steady release of mucins that provide a normal barrier.

Effective mucus clearance is essential for lung health, and airway disease is a consistent consequence of poor clearance. Healthy mucus is a gel with low viscosity and elasticity that is easily transported by ciliary action, whereas pathologic mucus has higher viscosity and elasticity and is less easily cleared (Cone 2009, Adv. Drug Deliv. Rev. 61:75-85; Innes at al. 2006, Chest 130:1102-1108). When mucin production is increased so that mucins accumulate intracellularly, and secretion of a large number of granules is then triggered (mucus hypersecretion), airway luminal occlusion can occur (Hayashi et al. 2004, Virchows Arch. 444:66-73; Hogg 1997, APMIS 105:735-745; Hays and Fahy 2003, Am. J. Med 115:68-69; Bossé et al. 2010, Annu. Rev. Physiol 72:437-462). The conversion from healthy to pathologic mucus occurs by multiple mechanisms that change its hydration and biochemical constituents; these include abnormal secretion of salt and water and increased production of mucins. The accumulation of mucus results from some combination of over production and decreased clearance, and persistent accumulation can lead to infection and inflammation by providing an environment for microbial growth.

The principal symptoms of impaired mucus clearance are cough and dyspnea. Cough is caused by the stimulation of vagal afferents in the intrapulmonary airways or the larynx and pharynx (Canning 2006, Chest 129: Suppl: 33S-47S; Rubin 2010, Lung 188: Suppl: S69-S72). Dyspnea is caused when mucus obstructs airflow by occupying the lumen of numerous airways (Hogg 2004, Lancet 364:709-721; Hogg 1997 supra; Hays and Fahy 2003 supra; Bossé et al. 2010 supra). Physical signs of impaired mucus clearance include cough, bronchial breath sounds, rhonchi, and wheezes. Untreated or untreatable airway mucus hypersecretion contributes significantly to patient morbidity and mortality not only due to the fact that excess mucus obstructs airways but because it contributes to airway hyperesponsiveness. Diseases which are characterised by mucus hypersecretion include asthma, cystic fibrosis, chronic obstructive pulmonary disease, immunodeficiency states (e.g. hypogammaglobulinemia, human immunodeficiency virus infection, organ transplantation, and hematologic malignant conditions). Retained mucus is also a problem in intubated patients and those in whom lung mechanics are disrupted as a result of paralysis, immobilization, or surgery; atelectasis and pneumonia are common complications in such patients.

All of these conditions are difficult to effectively treat and, currently, not curable. However, in terms of patient care and management, the development of means to effectively alleviate such a symptom is nevertheless highly desirable since it can significantly assist with ongoing disease management and thereby improve treatment outcomes. This will therefore necessarily greatly improve a patient's quality of life.

To date there has existed a limited understanding of the mechanisms underpinning mucus hypersecretion events. This has been significantly complicated by the wide range of different disease types, which all exhibit unique etiologies and mechanisms of action, with which mucus dysfunction is associated. In the context of some airway inflammatory conditions, for example, there occurs mucus hypersecretion and this symptom has therefore been considered in terms of whether it forms part of the inflammatory response and would be treatable by reducing inflammation. To date, however, simple anti-inflammatory treatment regimes have been of limited utility in this regard. To the extent that mucus hypersecretion occurs, however, any perceived link to the inflammatory cascade provides little assistance in relation to situations where the defect is in fact a reduced clearance mechanism rather than hypersecretion or where the hypersecretion occurs prior to inflammation events or in the context of entirely non-inflammatory conditions

These complexities have been reflected in the scientific literature where conflicting and vague data have been obtained. For example, in the context of Hardy et al., 2006 (Clin. Exp. Allergy 36:941-950), it was determined that follistatin treatment of a murine allergic asthma model appeared to result in a lower number of mucus producing airway cells. Bearing in mind that mice in fact do not naturally develop asthma, these results were of limited relevance, this in fact being reflected in the later publication of Hardy et al., 2010 (Am. J. Respir. Cell Molec. Biol. 42:667-675) where the authors state that subsequent studies by their group in fact showed no evidence for any direct link between activin A and mucus production in mouse lungs. This would imply that in this case activin A was not directly linked to mucus production and would discount a role for activin A in the context of mucus secretion and, further, regulation of mucus hypersecretion in the context of non-inflammatory responses. These findings were further supported by those of Gregory et al., 2010 (Am. J. Respir. Crit. Care Med 182:143-154) who directly investigated the effect of administering a neutralising antibody to activin A in the context of a dust mite mediated model of airway remodelling and hyperesponsiveness. In this study, these authors determined that there was no effect on epithelial mucus secretion by reducing levels of bioactive activin A by its binding to the antibodies. Accordingly, not only have there been findings that in the context of an inflammatory response the reduction of activin A does not directly impact on mucus secretion but, further, there has been no suggestion whatsoever as to how mucus secretion is regulated outside the context of inflammatory conditions or in the context of mucus dysfunction based on reduced clearance as opposed to aberrant hypersecretion.

In work leading up to the present invention, it has therefore been surprisingly determined that airway tissue mucus secretion, such as mucus hypersecretion, can be effectively reduced by either downregulating the levels of functional activin or increasing follistatin levels, irrespective of the co-existence of an inflammatory state. This finding has therefore provided a simple and efficient means to treat an extremely serious symptom which is characteristic of a broad range of diseases. Although not in itself a curative therapy for any of these diseases, alleviation of a symptom which is associated with extremely adverse complications and outcomes, irrespective of the nature of the cause of this symptom, is a significant step forward in terms of patient care and management.

### SUMMARY OF THE INVENTION

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

As used herein, the term “derived from” shall be taken to indicate that a particular integer or group of integers has originated from the species specified, but has not necessarily been obtained directly from the specified source. Further, as used herein the singular forms of “a”, “and” and “the” include plural referents unless the context clearly dictates otherwise.

Unless otherwise defined, all technical and scientific terms used herein have the same meaning as commonly understood by one of ordinary skill in the art to which this invention belongs.

One aspect of the present invention is directed to a method of reducing airway tissue mucus secretion in a mammal, said method comprising downregulating the functional level of activin in said mammal.

In another aspect, there is provided a method of reducing airway tissue mucus secretion in a mammal, said method comprising upregulating the functional level of follistatin.

In still another aspect there is provided a method of reducing lung tissue mucus secretion in a mammal, said method comprising downregulating the functional level of activin in said mammal.

In yet another aspect there is provided a method of reducing lung tissue mucus secretion in a mammal, said method comprising upregulating the functional level of follistatin in said mammal.

In a further aspect there is provided a method of reducing airway tissue mucus hypersecretion in a mammal, said method comprising downregulating the functional level of activin in said mammal.

In still another aspect, there is provided a method of reducing airway tissue mucus hypersecretion in a mammal, said method comprising upregulating the functional level of follistatin in said mammal.

In still yet another aspect the present invention provides a method of reducing airway tissue mucus secretion in a mammal, said method comprising downregulating the functional level of activin A or activin B in said mammal.

In yet still another aspect there is therefore provided a method of reducing airway tissue mucus secretion in a mammal, said method comprising administering to said mammal an effective amount of follistatin.

In another further aspect there is provided a method of reducing airway tissue mucus secretion in a mammal, said method comprising administering to said mammal an effective amount of inhibin for a time and under conditions sufficient to downregulate the functional level of activin in said mammal.

In a related aspect the present invention is directed to a method of therapeutically or prophylactically treating a condition which is characterised by airway tissue mucus dysfunction, said method comprising downregulating the functional level of activin in said mammal wherein downregulating said level of activin reduces airway tissue mucus secretion.

In a further aspect, the present invention is directed to a method of therapeutically or prophylactically treating a condition which is characterised by airway tissue mucus dysfunction, said method comprising upregulating the functional level of follistatin in said mammal wherein upregulating said level of follistatin reduces airway tissue mucus secretion.

In another further aspect the present invention is directed to a method of therapeutically or prophylactically treating cystic fibrosis in a mammal, said method comprising downregulating the functional level of activin or upregulating the functional level of follistatin in said mammal.

In still another further aspect there is provided a method of therapeutically or prophylactically treating asthma in a mammal, said method comprising downregulating the functional level of activin or upregulating the functional level of follistatin in said mammal.

In yet another further aspect there is provided a method of therapeutically or prophylactically treating chronic obstructive pulmonary disease in a mammal, said method comprising downregulating the functional level of activin or upregulating the functional level of follistatin in said mammal.

In still yet another aspect there is provided a method of therapeutically or prophylactically treating a mammal in which lung clearance mechanisms are disrupted, said method comprising downregulating the functional level of activin or upregulating the functional level of follistatin in said mammal.

Another aspect of the present invention relates to the use of an agent which downregulates the functional level of activin or upregulates the functional level of follistatin in the manufacture of a medicament for the treatment of a condition which is characterised by airway tissue mucus dysfunction.

### BRIEF DESCRIPTION OF THE DRAWINGS

FIG. 1: Airway mucus production in the lungs of Scnn1b transgenic mice. Lung sections were stained with Periodic Acid Schiff (PAS) and the degree of airway mucus production scored via double-blind analysis. 0=no airway mucus production, 1=infrequent airway mucus producing cells, 2=moderate airway mucus production with occasional luminal mucus, 3=mucus production in most airways, frequent luminal obstruction, to 4=severe mucus production and airway obstruction in most airways.

FIG. 2: Intranasal follistatin treatment of Scnn1b newborn mice decreases airway mucus production. Wild-type or Scnn1b mice received saline or follistatin intranasally (i.n.) every 2^{nd} day from 3-21 days of age. Mucus-producing cells are shown with a black arrow. Inflammatory cells are shown with a white arrow. PAS stain, original magnification 400×.

FIG. 3: Intranasal follistatin treatment of Scnn1b newborn mice decreases airway mucus production. Newborn mice were treated with follistatin or saline i.n. every 2^{nd} day from 3-21 days of age. Lung sections were stained with PAS stain and the degree of mucus production scored as per FIG. 1. Mean±standard error (sem). * P<0.05.

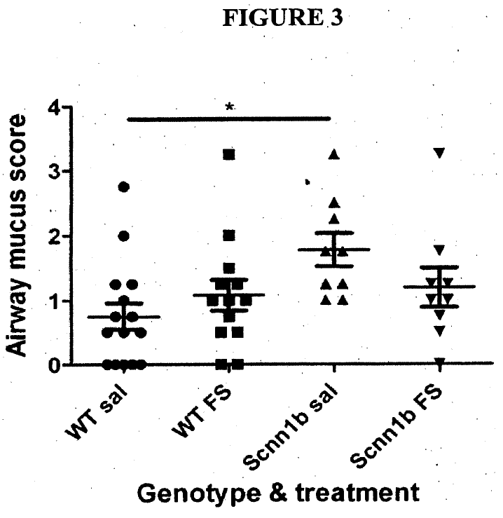

FIG. 4: Mean±sem IL-13 concentrations in BAL fluid of wild-type (WT) and Scnn1b mice, treated i.n. with isotonic saline (sal) or hrFS288 (FS). Bars indicate statistical significance between relevant groups; **P<0.01.

### DETAILED DESCRIPTION OF THE INVENTION

The present invention is predicated, in part, on the determination that airway tissue mucus secretion in a mammal can be reduced by either downregulating the level of functional activin or increasing the level of follistatin. Accordingly, this finding has facilitated the development of methods of prophylactically or therapeutically treating conditions characterised by airway tissue mucus dysfunction, such as mucus hypersecretion or decreased mucus clearance, and where a reduction in mucus secretion levels would alleviate the condition. Such conditions include, but are not limited to, asthma, cystic fibrosis, immunodeficiency conditions and conditions in which mucus is retained, such as intubation and paralysis.

Accordingly, one aspect of the present invention is directed to a method of reducing airway tissue mucus secretion in a mammal, said method comprising downregulating the functional level of activin in said mammal.

In another aspect, there is provided a method of reducing airway tissue mucus secretion in a mammal, said method comprising upregulating the functional level of follistatin.

In one embodiment said activin antagonist or follistatin levels are the levels in the airway tissue of said mammal.

By reference to “airway tissue” is meant the tissue of the passages which run from the mouth and nose, including the mouth and nose, into the lungs, together with the alveoli. The largest of the passages which runs from the oral and nasal cavities is the trachea (also known as the “windpipe”). In the chest, the trachea divides into two smaller passages termed the bronchi, each of these being further characterised by three regions termed the primary bronchus, secondary bronchus and tertiary bronchus. Each bronchus enters one lung and divides further into narrower passages termed the bronchioles. The terminal bronchiole supplies the alveoli. This network of passages is often colloquially termed the “bronchial tree”. Without limiting the present invention in any way, the predominant cell types in the pseudostratified columnar tracheal and bronchial epithelia include basal, intermediate, goblet, and ciliated cells. The simple columnar epithelia of bronchioles contain two main cell types termed Clara and ciliated cells. The most distal and functionally specialised epithelia of the lung include the gas exchanging air spaces; squamous type I pneumocytes and cuboidal type II pneumocytes.

In one embodiment, said airway tissue is lung tissue.

According to this embodiment there is provided a method of reducing lung tissue mucus secretion in a mammal, said method comprising downregulating the functional level of activin in said mammal.

In another embodiment there is provided a method of reducing lung tissue mucus secretion in a mammal, said method comprising upregulating the functional level of follistatin in said mammal.

Reference to “lung tissue” should be understood to include reference to the large airway passages which form part of the bronchial tree in each lung.

Reference to “mucus” should be understood as a reference to the viscous secretion, comprising mucins, which is secreted by mucosal tissue. Without limiting the present invention to any one theory or mode of action, airway luminal mucus is a complex dilute aqueous solution of lipids, glycoconjugates and proteins. It comprises salts, enzymes and anti-enzymes, oxidants and antioxidants, exogenous bacterial products, endogenous antibacterial agents, cell-derived mediators and proteins, plasma-derived mediators and proteins, and cell debris such as DNA. Airway mucus is considered to form a liquid bi-layer whereby an upper gel layer floats above a lower, more water soluble, or periciliary liquid, layer (Knowles and Boucher 2002, J. Clin. Invest. 109:571-577). Respiratory tract mucus requires the correct combination of viscosity and elasticity for optimal efficiency of ciliary interaction.

Viscoelasticity is conferred on the mucus primarily by high molecular weight mucous glycoproteins, termed mucins, which comprise up to 2% by weight of the mucus (Davies et al. 2002, Novartis Foundation Symposium 248. pp. 76-93). In the airways, mucins are produced by goblet cells in the epithelium (Rogers 2003, Int. J. Biochem. Cell. Biol. 35:1-6) and sero-mucous glands in the submucosal layer (Finkbeiner 1999, Respir. Physiol. 118:77-83). Mucins are thread-like molecules comprising a linear peptide sequence (termed apomucin), often with tandemly repeated regions, that is highly glycosylated, predominantly via O-linkages.

Reference to mucus “secretion” should therefore be understood as a reference to the secretion of mucus by the epithelial cells and sero-mucous glands in the submucosa of airway tissue. As detailed hereinbefore, mucus dysfunction is characterised by one or both of over production of mucus or decreased clearance of mucus. Reference to mucus “hypersecretion” should be understood as a reference to the overproduction of mucus, relative to normal levels of secretion, by the airway tissue.

Accordingly, in one embodiment there is provided a method of reducing airway tissue mucus hypersecretion in a mammal, said method comprising downregulating the functional level of activin in said mammal.

In still another embodiment, there is provided a method of reducing airway tissue mucus hypersecretion in a mammal, said method comprising upregulating the functional level of follistatin in said mammal.

In yet another embodiment, said airway tissue is lung tissue.

As detailed hereinbefore, irrespective of, and independently to, the existence or not of an inflammatory response, mucus dysfunction can occur. To date, where treatment for the disease condition as a whole is either ineffective or not known, there has been no known means of at least alleviating this very serious symptom. However, it has now been determined that airway tissue mucus secretion can be reduced by either downregulating the functional level of activin or upregulating the functional level of follistatin. Reference to mucus secretion being “reduced” should be understood as a reference to preventing, downregulating (e.g. slowing) or otherwise inhibiting mucus secretion. For example, this may take the form of reducing hypersecretion to restore normal levels of secretion or it may take the form of reducing normal levels of secretion. This latter outcome would be useful where the mucus dysfunction in a patient takes the form of impaired mucus clearance. In this situation, slowing secretion of mucus provides a means of reducing the rate of buildup and thereby enabling the reduced level of mucus clearance functionality to more effectively operate.

Reference to “activin” should be understood as a reference to an activin β subunit dimer. The subject dimer may be a homodimer or a heterodimer of the activin β subunits, these including β_{A}, β_{B}, β_{C} and β_{E}. Reference to the subunits should be understood to include reference to any isoforms which may arise from alternative splicing of activin β mRNA or mutant or polymorphic forms of activin β. Reference to “activin β” is not intended to be limiting and should be read as including reference to all forms of activin β including any protein encoded by the activin β subunit genes, any subunit polypeptide such as precursor forms which may be generated, and any β protein, whether existing as a monomer, multimer or fusion protein. Multimeric protein forms of activin include, for example, the homodimeric activin B (β_{B}-β_{B}) or the heterodimeric activin AB (β_{A}-β_{B}), activin BC (β_{B}-β_{C}), activin BE (β_{B}-β_{E}) activin A (β_{A}β_{A}), activin AC (β_{A}β_{C}), activin AE (β_{A}β_{E}), activin C (β_{C}β_{C}), activin CE (β_{C}β_{E}) and activin E (β_{E}β_{E}) proteins. Preferably, said activin molecule is activin A or activin B.

In accordance with this embodiment the present invention provides a method of reducing airway tissue mucus secretion in a mammal, said method comprising downregulating the functional level of activin A or activin B in said mammal.

In another embodiment, said airway tissue is lung tissue.

In still another embodiment, said mucus secretion is mucus hypersecretion.

Reference to “mammal” should be understood to include reference to a mammal such as but not limited to human, primate, livestock (animal (e.g. sheep, cow, horse, donkey, pig), companion animal (e.g. dog, cat), laboratory test animal (e.g. mouse, rabbit, rat, guinea pig, hamster), captive wild animal (e.g. fox, deer). Preferably the mammal is a human or primate. Most preferably the mammal is a human.

In terms of downregulating the “functional level” of activin or upregulating the “functional level” of follistatin, this should be understood to mean the level of activin or follistatin which is functional. It would be appreciated by the person of skill in the art that the functional level of activin can be downregulated either by reducing absolute levels of activin or by antagonising the functional activity of activin such that its effectiveness is decreased. Even the partial antagonism of activin may act to reduce, although not necessarily eliminate, the functional effectiveness of activin. Increasing the functional level of follistatin should be understood to have a converse meaning. For example one can increase the absolute levels of follistatin or one may increase its bioavailability, such as by increasing its half-life.

In terms of achieving the downregulation of activin, means for achieving this objective would be well known to the person of skill in the art and include, but are not limited to:
(i) Introducing into a cell a proteinaceous or non-proteinaceous molecule which downregulates the transcriptional and/or translational regulation of a gene, wherein this gene may be the activin gene or functional portion thereof or some other gene or gene region (e.g. promoter region) which directly or indirectly modulates the expression of the activin gene; or
(ii) Introducing a proteinaceous or non-proteinaceous molecule which functions as an antagonist to the activin expression product.

In terms of achieving upregulation of follistatin, this can also be achieved by any suitable method including administering the follistatin protein itself or introducing a proteinaceous or non-proteinaceous molecule which upregulates the transcription and/or translation of the follistatin gene.

The proteinaceous molecules described above may be derived from any suitable source such as natural, recombinant or synthetic sources and includes fusion proteins or molecules which have been identified following, for example, natural product screening. The reference to non-proteinaceous molecules may be, for example, a reference to a nucleic acid molecule or it may be a molecule derived from natural sources, such as for example natural product screening, or may be a chemically synthesised molecule. The present invention contemplates small molecules capable of acting as antagonists. Antagonists may be any compound capable of blocking, inhibiting or otherwise preventing activin from carrying out its normal biological function. Antagonists include monoclonal antibodies and antisense nucleic acids which prevent transcription or translation of activin genes or mRNA in mammalian cells. Modulation of expression may also be achieved utilising antigens, RNA, ribosomes, DNAzymes, aptamers, antibodies or molecules suitable for use in cosuppression. Suitable antisense oligonucleotide sequences (single stranded DNA fragments) of activin may be created or identified by their ability to suppress the expression of activin. The production of antisense oligonucleotides for a given protein is described in, for example, Stein and Cohen, 1988 (Cancer Res 48:2659-2668) and van der Krol et al., 1988 (Biotechniques 6:958-976). Antagonists also include any molecule that prevents activin interacting with its receptor.

In the context of antibodies, the present invention envisages the use of any suitable form of antibody including catalytic antibodies or derivatives, homologues, analogues or mimetics of said antibodies. Such antibodies may be monoclonal or polyclonal and may be selected from naturally occurring activin or its subunits or may be specifically raised to the activin dimer or its monomers (herein referred to as the “antigen”). In the case of the latter, the antigen may first need to be associated with a carrier molecule. Alternatively, fragments of antibodies may be used such as Fab fragments or Fab′₂ fragments. Furthermore, the present invention extends to recombinant and synthetic antibodies and to antibody hybrids. A “synthetic antibody” is considered herein to include fragments and hybrids of antibodies. The antigen can also be used to screen for naturally occurring antibodies.

Both polyclonal and monoclonal antibodies are obtainable by immunization with the antigen or derivative, homologue, analogue, mutant, or mimetic thereof and either type is utilizable therapeutically. The methods of obtaining both types of sera are well known in the art. Polyclonal sera are less preferred but are relatively easily prepared by injection of a suitable laboratory animal with an effective amount of the antigen, or antigenic parts thereof, collecting serum from the animal, and isolating specific sera by any of the known immunoabsorbent techniques. Although antibodies produced by this method are utilizable, they are generally less favoured because of the potential heterogeneity of the product.

The use of monoclonal antibodies is particularly preferred because of the ability to produce them in large quantities and the homogeneity of the product. The preparation of hybridoma cell lines for monoclonal antibody production derived by fusing an immortal cell line and lymphocytes sensitized against the immunogenic preparation can be done by techniques which are well known to those who are skilled in the art. (See, for example Douillard and Hoffman, 1981, in Compendium of Immunology); Köhler and Milstein, 1975 Nature 256: 495-497; Köhler and Milstein (1976) Eur. J. Immunol. 6: 511-519).

Preferably, the antibody of the present invention specifically binds the antigen. By “specifically binds” is meant high avidity and/or high affinity binding of an antibody to a specific antigen. Antibody binding to its epitope on this specific antigen is stronger than binding of the same antibody to any other epitope, particularly those that may be present in molecules in association with, or in the same sample, as the specific antigen of interest. Antibodies that bind specifically to a polypeptide of interest may be capable of binding other polypeptides at a weak, yet detectable, level (e.g., 10% or less of the binding shown to the polypeptide of interest). Such weak binding, or background binding, is readily discernible from the specific antibody binding to the polypeptide of interest, e.g. by use of appropriate controls.

The proteinaceous and non-proteinaceous molecules referred to, above, are herein collectively referred to as “modulatory agents”. To the extent that it is sought to decrease activin activity or increase follistatin activity, said modulatory agent is preferably:
(i) Follistatin. This may be administered either as a protein or its overexpression may be induced in vivo such as via the adenovirus mediated system described by Takabe et al., 2003 (Hepatology 38:1107-1115).
(ii) Any agent that upregulates the expression or functioning of the α subunit of inhibin. The α subunit can dimerise with the β subunits of activin to form inhibin, thereby effectively downregulating activin levels.
(iii) Inhibin. This molecule can bind to β-glycan and inhibit the actions of activin via its receptor. See for example the mechanism described by Xu et al., 1995 (J. Biol. Chem. 270:6308-6313) or the use of the Smad7 antagonist (Bernard et al., 2004; Molec. Endocrinol. 18:606-623).
(iv) Any agent that upregulates levels of β_{C} since this results in the formation of the inactive AC form of activin.
(v) Activin neutralising antibody. For example, as described in Poulaki et al., 2004 (Am. J. Pathol. 164:1293-1302).
(vi) Activin mutants which inhibit native activin from binding to its receptor. For example, as described in Harrison et al., 2004 (J. Biol. Chem. 279:28036-28044).
(vii) Transfection or treatment with a mutant activin receptor which prevents normal activin signalling or a soluble activin receptor which acts as a competitive inhibitor. See for example, the system described by Maeshima at al., 2004 (Endocrinology 145:3739-3745).
(viii) An activin antisense oligonucleotide.

In this regard, reference to “follistatin” should be read as including reference to all forms of follistatin including, by way of example, the three protein cores and six molecular weight forms which have been identified as arising from the alternatively spliced mRNAs FS315 and FS288. Accordingly, it should also be understood to include reference to any isoforms which may arise from alternative splicing of follistatin mRNA or mutant or polymorphic forms of follistatin. It should still further be understood to extend to any protein encoded by the follistatin gene, any subunit polypeptide, such as precursor forms which may be generated, and any follistatin protein or functional fragment, whether existing as a monomer, multimer or fusion protein. An analogous definition applies to “inhibin”.

Screening for the modulatory agents hereinbefore defined can be achieved by any one of several suitable methods including, but in no way limited to, contacting a cell comprising the activin gene or functional equivalent or derivative thereof with an agent and screening for the downregulation of activin protein production or functional activity, downregulation of the expression of a nucleic acid molecule encoding activin or downregulation of the activity or expression of downstream activin cellular target. Detecting such downregulation can be achieved utilising techniques such as Western blotting, electrophoretic mobility shift assays and/or the readout of reporters of activin activity such as luciferases, CAT and the like.

It should be understood that the activin gene or functional equivalent or derivative thereof may be naturally occurring in the cell which is the subject of testing or it may have been transfected into a host cell for the purpose of testing. Further, the naturally occurring or transfected gene may be constitutively expressed—thereby providing a model useful for, inter alia, screening for agents which down regulate activin activity, at either the nucleic acid or expression product levels, or the gene may require activation—thereby providing a model useful for, inter alia, screening for agents which up-regulate activin expression. Further, to the extent that an activin nucleic acid molecule is transfected into a cell, that molecule may comprise the entire activin gene or it may merely comprise a portion of the gene such as the portion which regulates expression of the activin product. For example, the activin promoter region may be transfected into the cell which is the subject of testing. In this regard, where only the promoter is utilised, detecting modulation of the activity of the promoter can be achieved, for example, by ligating the promoter to a reporter gene. For example, the promoter may be ligated to luciferase or a CAT reporter, the downregulation of expression of which gene can be detected via modulation of fluorescence intensity or CAT reporter activity, respectively. In another example, the subject of detection could be a downstream activin regulatory target, rather than activin itself. Yet another example includes activin binding sites ligated to a minimal reporter.

These methods provide a mechanism for performing high throughput screening of putative modulatory agents such as the proteinaceous or non-proteinaceous agents comprising synthetic, combinatorial, chemical and natural libraries. These methods will also facilitate the detection of agents which bind either the activin nucleic acid molecule or expression product itself or which modulate the expression of an upstream molecule, which upstream molecule subsequently downregulates activin expression or expression product activity. Accordingly, these methods provide a mechanism of detecting agents which either directly or indirectly modulate activin expression and/or activity.

The agents which are utilised in accordance with the method of the present invention may take any suitable form. For example, proteinaceous agents may be glycosylated or unglycosylated, phosphorylated or dephosphorylated to various degrees and/or may contain a range of other molecules used, linked, bound or otherwise associated with the proteins such as amino acids, lipid, carbohydrates or other peptides, polypeptides or proteins. Similarly, the subject non-proteinaceous molecules may also take any suitable form. Both the proteinaceous and non-proteinaceous agents herein described may be linked, bound otherwise associated with any other proteinaceous or non-proteinaceous molecules. For example, in one embodiment of the present invention said agent is associated with a molecule which permits its targeting to a localised region.

The subject proteinaceous or non-proteinaceous molecule may act either directly or indirectly to downregulate the expression of activin or the activity of the activin expression product. Said molecule acts directly if it associates with the activin nucleic acid molecule or expression product to modulate expression or activity, respectively. Said molecule acts indirectly if it associates with a molecule other than the activin nucleic acid molecule or expression product which other molecule either directly or indirectly downregulates the expression or activity of the activin nucleic acid molecule or expression product, respectively. Accordingly, the method of the present invention encompasses the regulation of activin nucleic acid molecule expression or expression product activity via the induction of a cascade of regulatory steps.

The term “expression” refers to the transcription and translation of a nucleic acid molecule. Reference to “expression product” is a reference to the product produced from the transcription and translation of a nucleic acid molecule.

“Derivatives” of the molecules herein described (for example activin A, activin B, follistatin or other proteinaceous or non-proteinaceous agents) include fragments, parts, portions or variants from either natural or non-natural sources. Non-natural sources include, for example, recombinant or synthetic sources. By “recombinant sources” is meant that the cellular source from which the subject molecule is harvested has been genetically altered. This may occur, for example, in order to increase or otherwise enhance the rate and volume of production by that particular cellular source. Parts or fragments include, for example, active regions of the molecule. Derivatives may be derived from insertion, deletion or substitution of amino acids. Amino acid insertional derivatives include amino and/or carboxylic terminal fusions as well as intrasequence insertions of single or multiple amino acids. Insertional amino acid sequence variants are those in which one or more amino acid residues are introduced into a predetermined site in the protein although random insertion is also possible with suitable screening of the resulting product. Deletional variants are characterised by the removal of one or more amino acids from the sequence. Substitutional amino acid variants are those in which at least one residue in a sequence has been removed and a different residue inserted in its place. Additions to amino acid sequences include fusions with other peptides, polypeptides or proteins, as detailed above.

Derivatives also include fragments having particular epitopes or parts of the entire protein fused to peptides, polypeptides or other proteinaceous or non-proteinaceous molecules. For example, follistatin, or derivative thereof may be fused to a molecule to facilitate its localisation to a particular site. Analogues of the molecules contemplated herein include, but are not limited to, modification to side chains, incorporating of unnatural amino acids and/or their derivatives during peptide, polypeptide or protein synthesis and the use of crosslinkers and other methods which impose conformational constraints on the proteinaceous molecules or their analogues.

Derivatives of nucleic acid sequences which may be utilised in accordance with the method of the present invention may similarly be derived from single or multiple nucleotide substitutions, deletions and/or additions including fusion with other nucleic acid molecules. The derivatives of the nucleic acid molecules utilised in the present invention include oligonucleotides, PCR primers, antisense molecules, molecules suitable for use in cosuppression and fusion of nucleic acid molecules. Derivatives of nucleic acid sequences also include degenerate variants.

A “variant” or “mutant” should be understood to mean molecules which exhibit at least some of the functional activity of the form of molecule (e.g. follistatin) of which it is a variant or mutant. A variation or mutation may take any form and may be naturally or non-naturally occurring.

A “homologue” is meant that the molecule is derived from a species other than that which is being treated in accordance with the method of the present invention. This may occur, for example, where it is determined that a species other than that which is being treated produces a form of follistatin, for example, which exhibits similar and suitable functional characteristics to that of the follistatin which is naturally produced by the subject undergoing treatment.

Chemical and functional equivalents should be understood as molecules exhibiting any one or more of the functional activities of the subject molecule, which functional equivalents may be derived from any source such as being chemically synthesised or identified via screening processes such as natural product screening. For example chemical or functional equivalents can be designed and/or identified utilising well known methods such as combinatorial chemistry or high throughput screening of recombinant libraries or following natural product screening. Antagonistic agents can also be screened for utilising such methods.

For example, libraries containing small organic molecules may be screened, wherein organic molecules having a large number of specific parent group substitutions are used. A general synthetic scheme may follow published methods (e.g., Bunin et al. (1994) Proc. Natl. Acad. Sci. USA, 91:4708-4712; DeWitt et al. (1993) Proc. Natl. Acad. Sci. USA, 90:6909-6913). Briefly, at each successive synthetic step, one of a plurality of different selected substituents is added to each of a selected subset of tubes in an array, with the selection of tube subsets being such as to generate all possible permutation of the different substituents employed in producing the library. One suitable permutation strategy is outlined in U.S. Pat. No. 5,763,263.

There is currently widespread interest in using combinational libraries of random organic molecules to search for biologically active compounds (see for example U.S. Pat. No. 5,763,263). Ligands discovered by screening libraries of this type may be useful in mimicking or blocking natural ligands or interfering with the naturally occurring ligands of a biological target. By use of techniques, such as that disclosed in U.S. Pat. No. 5,753,187, millions of new chemical and/or biological compounds may be routinely screened in less than a few weeks. Of the large number of compounds identified, only those exhibiting appropriate biological activity are further analysed.

With respect to high throughput library screening methods, oligomeric or small-molecule library compounds capable of interacting specifically with a selected biological agent, such as a biomolecule, a macromolecule complex, or cell, are screened utilising a combinational library device which is easily chosen by the person of skill in the art from the range of well-known methods, such as those described above. In such a method, each member of the library is screened for its ability to interact specifically with the selected agent. In practising the method, a biological agent is drawn into compound-containing tubes and allowed to interact with the individual library compound in each tube. The interaction is designed to produce a detectable signal that can be used to monitor the presence of the desired interaction. Preferably, the biological agent is present in an aqueous solution and further conditions are adapted depending on the desired interaction. Detection may be performed for example by any well-known functional or non-functional based method for the detection of substances.

In one embodiment, downregulation of the functional level of activin is achieved by administering follistatin, inhibin, an antibody directed to activin, an activin antisense oligonucleotide, a non-functional activin molecule which competitively inhibits binding to the activin receptor or a mutant or soluble activin receptor which inhibits normal activin signalling.

Accordingly, in one particular embodiment there is therefore provided a method of reducing airway tissue mucus secretion in a mammal, said method comprising administering to said mammal an effective amount of follistatin.

In relation to this particular embodiment, it should be understood that in the context of some conditions follistatin may function to reduce mucus secretion by inhibiting activin functionality while in other conditions it may function independently to activin. Without limiting the present invention to any one theory or mode of action, follistatin is a blocker of other TGFβ members, and can, independently of activin, reduce mucus secretion. This therefore provides a valuable means of reducing mucus secretion in conditions beyond just those where mucus secretion is regulated by activin.

In another particular embodiment there is provided a method of reducing airway tissue mucus secretion in a mammal, said method comprising administering to said mammal an effective amount of inhibin for a time and under conditions sufficient to downregulate the functional level of activin in said mammal.

As detailed hereinbefore, a further aspect of the present invention relates to the use of the invention in relation to the treatment and/or prophylaxis of disease conditions or other unwanted conditions which are characterised by mucus dysfunction.

Accordingly, in a related aspect the present invention is directed to a method of therapeutically or prophylactically treating a condition which is characterised by airway tissue mucus dysfunction, said method comprising downregulating the functional level of activin in said mammal wherein downregulating said level of activin reduces airway tissue mucus secretion.

In a further aspect, the present invention is directed to a method of therapeutically or prophylactically treating a condition which is characterised by airway tissue mucus dysfunction, said method comprising upregulating the functional level of follistatin in said mammal wherein upregulating said level of follistatin reduces airway tissue mucus secretion.

Reference to “mucus dysfunction” should be understood as a reference to either secreted mucus levels which are higher than normal levels or else, irrespective of what level of mucus is secreted, decreased mucus clearance functionality. In both these situations a buildup of mucus occurs in the airways, this having extremely serious implications for the patient. Reference to a “condition characterised by mucus dysfunction” should therefore be understood as a reference to any condition, a symptom or cause of which is airway tissue mucus dysfunction. To this end, it should be understood that this extends to conditions in respect of which mucus secretion and clearance is normal but may nevertheless be unwanted or otherwise problematic. Examples of such conditions include, but are not limited to, asthma, cystic fibrosis, chronic obstructive pulmonary disease, bronchiectasis, primary ciliary dyskinesia, panbronchiolitis, chronic bronchitis, pulmonary hypertension, idiopathic pulmonary fibrosis, immunodeficiency states (e.g. hypogammaglobulinemia, human immunodeficiency virus infection, organ transplantation, and hematologic malignant conditions), intubated patients, impaired mucus clearance, and those in whom lung mechanics are disrupted as a result of paralysis immobilization or surgery.

Accordingly, in one embodiment the present invention is directed to a method of therapeutically or prophylactically treating cystic fibrosis in a mammal, said method comprising downregulating the functional level of activin or upregulating the functional level of follistatin in said mammal.

In another embodiment there is provided a method of therapeutically or prophylactically treating asthma in a mammal, said method comprising downregulating the functional level of activin or upregulating the functional level of follistatin in said mammal.

In yet another embodiment there is provided a method of therapeutically or prophylactically treating chronic obstructive pulmonary disease in a mammal, said method comprising downregulating the functional level of activin or upregulating the functional level of follistatin in said mammal.

In still yet another embodiment there is provided a method of therapeutically or prophylactically treating a mammal in which lung clearance mechanisms are disrupted, said method comprising downregulating the functional level of activin or upregulating the functional level of follistatin in said mammal.

According to this embodiment, said lung clearance mechanisms are disrupted due to intubation, paralysis, surgery or immobilisation.

In still another embodiment, said condition is:
a non-inflammatory condition;
one in which unwanted mucus secretion or mucus hypersecretion occurs prior to the onset of inflammation or is regulated by non-inflammatory mechanisms; or
one in which mucus secretion levels are unchanged from normal levels but are unwanted and sought to be reduced, whether that be in the context of either an inflammatory or non-inflammatory condition.

In accordance with the embodiments, said airway tissue is lung tissue.

In another embodiment, said activin is activin A or activin B.

The agent which is administered to downregulate activin functionality is administered in an amount necessary at least partly to attain the desired response, or to delay the onset or inhibit progression or halt altogether, the onset or progression of the particular condition being treated. The amount varies depending upon the health and physical condition of the individual to be treated, the taxonomic group of the individual to be treated, the degree of protection desired, the formulation of the composition, the assessment of the medical situation, and other relevant factors. It is expected that the amount will fall in a relatively broad range that can be determined through routine trials.

In yet another embodiment downregulation of the functional level of activin is achieved by administering follistatin, inhibin, an antibody directed to activin, an activin antisense molecule, a non-functional activin molecule which competitively inhibits binding to the activin receptor or a mutant or soluble activin receptor which inhibits normal activin signalling.

Reference herein to “treatment” and “prophylaxis” is to be considered in its broadest context. The term “treatment” does not necessarily imply that a subject is treated until total recovery. Similarly, “prophylaxis” does not necessarily mean that the subject will not eventually contract a disease condition. Accordingly, treatment and prophylaxis include amelioration of the symptoms of a particular condition or preventing or otherwise reducing the risk of developing a particular condition. The term “prophylaxis” may be considered as reducing the severity or onset of a particular condition. “Treatment” may also reduce the severity of an existing condition.

The present invention further contemplates a combination of therapies, such as the administration of the modulatory agent together with other proteinaceous or non-proteinaceous molecules which may facilitate the desired therapeutic or prophylactic outcome. For example, one may combine the method of the present invention with standard asthma or cystic fibrosis treatment regimes.

Administration of molecules of the present invention hereinbefore described [herein collectively referred to as “modulatory agent” ], in the form of a pharmaceutical composition, may be performed by any convenient means. The modulatory agent of the pharmaceutical composition is contemplated to exhibit therapeutic activity when administered in an amount which depends on the particular case. The variation depends, for example, on the human or animal and the modulatory agent chosen. A broad range of doses may be applicable. Considering a patient, for example, from about 0.1 μg to about 1 mg of modulatory agent may be administered per kilogram of body weight per day. Dosage regimes may be adjusted to provide the optimum therapeutic response. For example, several divided doses may be administered daily, weekly, monthly or other suitable time intervals or the dose may be proportionally reduced as indicated by the exigencies of the situation.

The modulatory agent may be administered in a convenient manner such as by the oral, intravenous (where water soluble), respiratory, transdermal, intraperitoneal, intramuscular, subcutaneous, intradermal or suppository mutes or implanting (e.g. using slow release molecules). The modulatory agent may be administered in the form of pharmaceutically acceptable nontoxic salts, such as acid addition salts or metal complexes, e.g. with zinc, iron or the like (which are considered as salts for purposes of this application). Illustrative of such acid addition salts are hydrochloride, hydrobromide, sulphate, phosphate, maleate, acetate, citrate, benzoate, succinate, malate, ascorbate, tartrate and the like. If the active ingredient is to be administered in tablet form, the tablet may contain a binder such as tragacanth, corn starch or gelatin; a disintegrating agent, such as alginic acid; and a lubricant, such as magnesium stearate.

Routes of administration include, but are not limited to, systemically, locally, respiratorally, transdermally, intratracheally, nasopharyngeally, intravenously, intraperitoneally, subcutaneously, intracranially, intradermally, intramuscularly, intraoccularly, intrathecally, intracerebrally, intranasally, infusion, orally, rectally, via IV drip, patch and implant. Preferably, said means of administration is inhalation with respect to the treatment of airway mucus secretion and intravenously, intramuscularly or transdermally for other conditions.

The modulatory agent may be administered in any convenient or suitable manner although respiratory routes are preferred. For example, one may administer by inhalation or insufflation of powders or aerosols (including by nebulizer); intratracheal or intranasal.

For inhalation, the composition of the invention can be delivered using any system known in the art, including dry powder aerosols, liquids delivery systems, air jet nebulizers, propellant systems, and the like. See, e.g., Patton (1998) Biotechniques 16:141-143; product and inhalation delivery systems for polypeptide macromolecules by, e.g., Dura Pharmaceuticals (San Diego, Calif.), Aradigm (Hayward, Calif.), Aerogen (Santa Clara, Calif.), Inhale Therapeutic Systems (San Carlos, Calif.), PARI Pharma (Graefelfing, Germany) and the like. For example, the pharmaceutical formulation can be administered in the form of an aerosol or mist. For aerosol administration, the formulation can be supplied in finely divided form along with a surfactant and propellant. In another aspect, the device for delivering the formulation to respiratory tissue is an inhaler in which the formulation vaporizes. Other liquid delivery systems include, e.g., air jet nebulizers. In yet another aspect, the formulation can be administered as a dry spray.

In one embodiment, said activin antagonist or follistatin is administered systemically.

In another embodiment, said activin antagonist or follistatin administration is localised to the airway, in particular the lung, for example by inhalation through the nose and/or mouth of aerosol or via a liquid delivery system or nebulizer.

In accordance with these methods, the agent defined in accordance with the present invention may be coadministered with one or more other compounds or molecules. By “coadministered” is meant simultaneous administration in the same formulation or in two different formulations via the same or different routes or sequential administration by the same or different routes. For example, the subject agent may be administered together with an agonistic agent in order to enhance its effects. By “sequential” administration is meant a time difference of from seconds, minutes, hours or days between the administration of the two types of molecules. These molecules may be administered in any order.

Another aspect of the present invention relates to the use of an agent which downregulates the functional level of activin or upregulates the functional level of follistatin in the manufacture of a medicament for the treatment of a condition which is characterised by airway tissue mucus dysfunction.

In one embodiment, said condition is asthma, cystic fibrosis, chronic obstructive pulmonary disease, bronchiectasis, primary ciliary dyskinesia, pulmonary hypertension, immunodeficiency states (e.g. hypogammaglobulinemia, human immunodeficiency virus infection, organ transplantation, and hematologic malignant conditions), intubated patients and those in whom lung mechanics are disrupted as a result of paralysis, immobilization or surgery.

In another embodiment, said activin is activin A or activin B.

In yet another embodiment downregulation of the functional level of activin is achieved by administering follistatin, inhibin, an antibody directed to activin, an activin antisense molecule, a non-functional activin molecule which competitively inhibits binding to the activin receptor or a mutant or soluble activin receptor which inhibits normal activin signalling.

The pharmaceutical forms suitable for injectable use include sterile aqueous solutions (where water soluble) or dispersions and sterile powders for the extemporaneous preparation of sterile injectable solutions or dispersion or may be in the form of a cream or other form suitable for topical application. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol and liquid polyethylene glycol, and the like), suitable mixtures thereof, and vegetable oils. The proper fluidity can be maintained, for example, by the use of a coating such as lecithin, by the maintenance of the required particle size in the case of dispersion and by the use of superfactants. The preventions of the action of microorganisms can be brought about by various antibacterial and antifungal agents, for example, parabens, chlorobutanol, phenol, sorbic acid, thimerosal and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compounds in the required amount in the appropriate solvent with various of the other ingredients enumerated above, as required, followed by filtered sterilisation. Generally, dispersions are prepared by incorporating the various sterilised active ingredient into a sterile vehicle which contains the basic dispersion medium and the required other ingredients from those enumerated above. In the case of sterile powders for the preparation of sterile injectable solutions, the preferred methods of preparation are vacuum drying and the freeze-drying technique which yield a powder of the active ingredient plus any additional desired ingredient from previously sterile-filtered solution thereof.

When the active ingredients are suitably protected they may be orally administered, for example, with an inert diluent or with an assimilable edible carrier, or it may be enclosed in hard or soft shell gelatin capsule, or it may be compressed into tablets, or it may be incorporated directly with the food of the diet. For oral therapeutic administration, the active compound may be incorporated with excipients and used in the form of ingestible tablets, buccal tablets, troches, capsules, elixirs, suspensions, syrups, wafers, and the like. Such compositions and preparations should contain at least 1% by weight of active compound. The percentage of the compositions and preparations may, of course, be varied and may conveniently be between about 5 to about 80% of the weight of the unit. The amount of active compound in such therapeutically useful compositions in such that a suitable dosage will be obtained. Preferred compositions or preparations according to the present invention are prepared so that an oral dosage unit form contains between about 0.1 μg and 2000 mg of active compound.

The agent may also be prepared for administration via the airway in either a particulate or soluble form. For example, the agent may be administered via an oral inhaler or a nebuliser.

The tablets, troches, pills, capsules and the like may also contain the components as listed hereafter a binder such as gum, acacia, corn starch or gelatin; excipients such as dicalcium phosphate; a disintegrating agent such as corn starch, potato starch, alginic acid and the like; a lubricant such as magnesium stearate; and a sweetening agent such as sucrose, lactose or saccharin may be added or a flavouring agent such as peppermint, oil of wintergreen, or cherry flavouring. When the dosage unit form is a capsule, it may contain, in addition to materials of the above type, a liquid carrier. Various other materials may be present as coatings or to otherwise modify the physical form of the dosage unit. For instance, tablets, pills, or capsules may be coated with shellac, sugar or both. A syrup or elixir may contain the active compound, sucrose as a sweetening agent, methyl and propylparabens as preservatives, a dye and flavouring such as cherry or orange flavour. Of course, any material used in preparing any dosage unit form should be pharmaceutically pure and substantially nontoxic in the amounts employed. In addition, the active compound(s) may be incorporated into sustained-release preparations and formulations.

The pharmaceutical composition may also comprise genetic molecules such as a vector capable of transfecting target cells where the vector carries a nucleic acid molecule encoding follistatin or a modulatory agent as hereinbefore defined. The vector may, for example, be a viral vector.

The present invention is further described by reference to the following non-limiting examples.

### Example 1

### Breeding and Characterisation of Cystic Fibrosis Mice

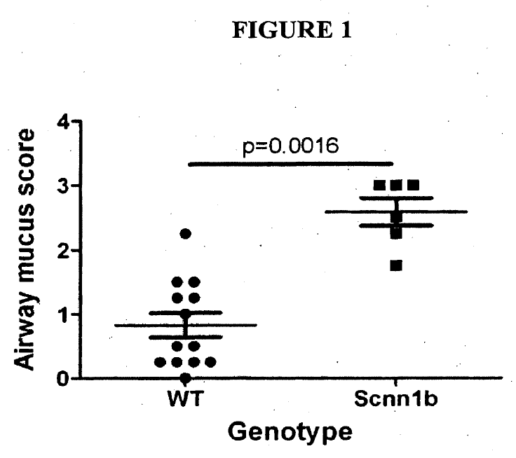

The Scnn1b (also known as βENaC) transgenic mice, which develop cystic fibrosis-like disease, were successfully imported, and mated to a second line of mice (a cross between C57BL/6×C3H/HeJ strains). Both lines bred well. Scnn1b mice develop the expected phenotype, with 40-50% of transgenic mice dying by 21 days of age. Scnn1b mice also show the expected lung pathology (Mall et al., 2004, Nature Med. 10:487-493), with excessive mucus production in the lung airways as reflected in an increased mucus production score compared to normal mice (FIG. 1).

### Effect of Follistatin Treatment on Lung Disease

Litters of newborn mice were randomly assigned to either follistatin treatment or saline control groups. Mouse pups received follistatin or saline via the intranasal route, every 2^{nd} day, from 3-21 days of age. A dose of 250 μg/kg was used throughout the studies described herein. Mice were weighed daily, and the follistatin concentration and volume adjusted accordingly. Pups that had survived until Days 21-23 or age were killed humanely by CO2 asphyxiation. Thereafter, blood was collected from the inferior vena cava. Serum was obtained from whole blood by centrifugation for 4 minutes at 11,350 g and samples were stored at −20′C.

Bronchioalveolar lavage (BAL) fluid was collected by lavaging the airways with 0.3 mL of 1% fetal calf serum in phosphate-buffered saline (PBS), followed by three further lavages of 0.2 mL, to give a total BAL fluid sample of ˜0.9 mL per animal. BAL samples were centrifuged at 350 g for 4 minutes, and stored at −70° C. for subsequent cytokine/chemokine analysis.

After processing for BAL fluid, lungs were removed and placed into freshly-made neutral buffered formalin. Formalin-fixed lungs were paraffin-embedded and 3 μm sections were cut. These were stained with periodic acid-Schiff (PAS) for analysis of goblet cells and presence of mucus. The degree of PAS staining, indicative of mucus production and goblet cells was scored by double-blind analysis (two independent operators). A qualitative score for each lung was derived using the following scores 0=no airway mucus production. 1=infrequent airway mucus-producing cells, 2=moderate airway mucus production with occasional luminal mucus, 3=mucus production in most airways, frequent luminal obstruction, to 4=severe mucus production and airway obstruction in most airways.

Various chemokine and cytokine concentrations in BAL fluid samples were determined using a mouse 23-plex assey kit (Bio-Rad; http://www.bio-rad.com/prd/en/US/LSR/SKU/M60-009RDPD/Bio-Plex_Protrade_Mouse_Cytokine_23-plex_Assay). This kit measures a number of chemokines and cytokines including IL-13.

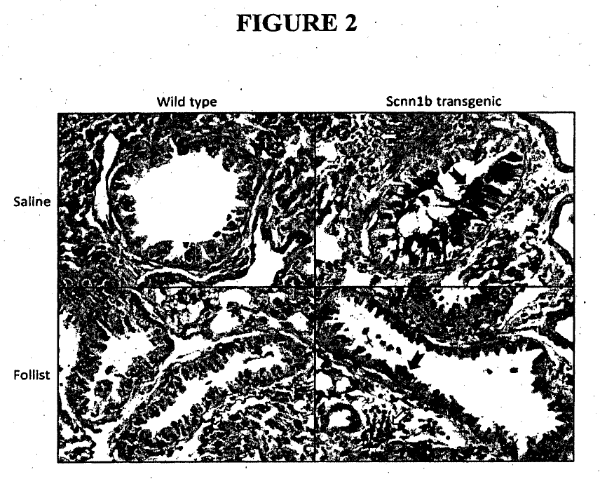

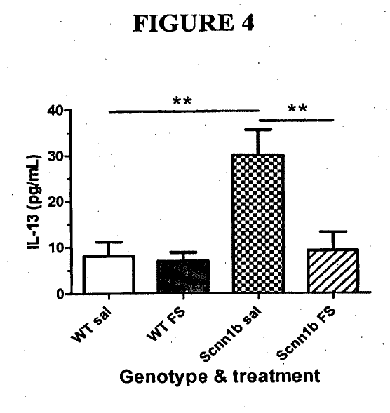

Cystic fibrosis patients produce excessive mucus in the lungs, leading to obstruction of the airways and loss of lung function, a finding mirrored in Scnn1b mice as described above (FIG. 1). The effect of follistatin treatment on mucus production was assessed by staining lung sections with periodic acid Schiff (PAS), and performing semi-quantitative double blind analysis of the degree of mucus production. Wild-type mice had a generally low level of mucus production (FIGS. 2 & 3). Importantly, the high level of mucus production seen in saline-treated Scnn1b mice was markedly decreased by follistatin treatment (FIGS. 2 & 3). When BAL fluid concentrations of IL13 were assessed, there was no effect of follistatin administration in wild-type animals (FIG. 4). Scnn1b mice had significantly elevated IL13 concentrations in BAL fluid, and follistatin treatment of Scnn1b mice led to a significant reduction in IL13 concentrations back to levels consistent with those seen in wild-type mice (FIG. 4).

### Effect of Follistatin Treatment on Lifespan and General Well-being

Cystic fibrosis patients have markedly reduced life-span, a feature also observed in Scnn1b mice. Importantly, while 25% of mice in the saline treatment group (n=24 total) died by 21 day of age, this figure was 18% in the follistatin treatment group (n=22 total). These data indicate that follistatin increases overall survival. Another important finding is that follistatin dosing every second day for 3 weeks does not cause lung pathology or ill health.

Those skilled in the art will appreciate that the invention described herein is susceptible to variations and modifications other than those specifically described. It is to be understood that the invention includes all such variations and modifications. The invention also includes all of the steps, features, compositions and compounds referred to or indicated in this specification, individually or collectively, and any and all combinations of any two or more of said steps or features.

### BIBLIOGRAPHY

Adler and Li (2001) Airway epithelium and mucus: intracellular signaling pathways for gene expression and secretion. Am. J. Respir. Cell Molec. Biol. 25:397-400
Bernard et al. (2004) Both SMAD2 and SMAD3 mediate activin-stimulated expression of the follicle-stimulating hormone β subunit in mouse gonadotrope cells. Mol. Endocrinol. 18:606-623
Bossé et al. (2010) It's not all smooth muscle: non-smooth-muscle elements in control of resistance to airflow. Annu. Rev. Physiol. 72:437-462
Bunin et al. (1994) The combinatorial synthesis and chemical and biological evaluation of a 1,4-benzodiazepine library. Proc. Natl. Acad Sci. USA, 91:4708-4712
Canning (2006) Anatomy and neurophysiology of the cough reflex: ACCP Evidence-Based Clinical Practice Guidelines. Chest 129 (Suppl.): 33S-47S
Cone (2009) Barrier properties of mucus. Adv. Drug Deliv. Rev. 61:75-85
Davies et al. (2002) ‘Respiratory tract mucins: structure and expression patterns’ In Mucus Hypersecretion in Respiratory Disease. Novartis Foundation Symposium 248, pp. 76-93
Davis and Dickey (2008) Regulated airway goblet cell mucin secretion. Annu. Rev. Physiol. 70:487-512
Davis and Lazarowski (2008) Coupling of airway ciliary activity and mucin secretion to mechanical stresses by purinergic signalling. Respir. Physiol. Neurobiol. 163:208-213
DeWitt et al. (1993) “Diversomers”: an approach to nonpeptide, nonoligomeric chemical diversity. Proc. Natl. Acad. Sci. USA, 90:6909-6913
Douillard and Hoffman (1981) Basic Facts about Hybridomas, in Compendium of Immunology Vol II, ed. by Schwartz, 1981
Finkbeiner (1999) Physiology and pathology of tracheobronchial glands. Respir. Physiol. 118:77-83
Gregory et al. (2010) Overexpression of Smad2 drives house dust mite-mediated airway remodelling and airway hyperresponsiveness via activin and IL25. Am. J. Respir. Crit. Care Med 182:143-154
Hardy et al. (2006) Follistatin is a candidate endogenous negative regulator of activin A in experimental allergic asthma. Clin. Exp. Allergy 36: 941-950
Hardy et al. (2010) Interleukin-13 regulates secretion of the tumor growth factor-β superfamily cytokine activin A in allergic asthma inflammation. Am. J. Respir. Cell Molec. Biol. 42:667-675
Harrison et al. (2004) An activin mutant with disrupted ALK4 binding blocks signaling via type II receptors. J. Biol. Chem. 279:28036-28044
Hayashi et al. (2004) Ultrastructure of goblet-cell metaplasia from Clara cell in the allergic asthma inflammation in a mouse model of asthma in vivo. Virchows Arch. 444:66-73
Hays and Fahy (2003) The role of mucus in fatal asthma. Am. J. Med 115:68-69
Hogg (1997) The pathology of asthma. APMIS 105:735-745
Hogg (2004) Pathophysiology of airflow limitation in chronic obstructive pulmonary disease. Lancet 364:709-721
Innes et al. (2006) Epithelial mucus scores are increased in the large airways of smokers with airflow obstruction. Chest 130:1102-1108
Kim et al. (2003) Pharmacology of airway goblet cell mucin release. J. Pharmacol. Sci, 92:301-307
Knowles and Boucher (2002) Mucus clearance as a primary innate defence mechanism for mammalian airways. J. Clin. Invest. 109:571-577
Köhler and Milstein (1975) Continuous cultures of fused cells secreting antibody of predefined specificity. Nature 256: 495-497, Köhler and Milstein (1976) Derivation of specific antibody-producing tissue culture and tumor lines by cell fusion. Eur. J. Immunol. 6:511-519
Lazarowski and Boucher (2009) Purinergic receptors in airway epithelia. Curr. Opin. Pharmacol. 9:262-267
Maeshima at al. (2004) Crucial role of activin A in tubulogenesis of endothelial cells induced by vascular endothelial growth factor. Endocrinology 145:3739-3745
Mall et al. (2004) Increased airway epithelial Na⁺ absorption produces cystic fibrosis-like lung disease in mice. Nature Med. 10:487-493
Patton (1998) BioTechniques 16:141-143
Poulaki et al. (2004) Activin A in the regulation of corneal vascularization and vascular endothelial growth factor expression. Am. J. Pathol. 164:1293-1302
Rogers (2003) The airway goblet cell. Int. J. Biochem. Cell Biol. 35:1-6
Rubin (2010) The role of mucus in cough research. Lung 188 (Suppl.): S69-S72
Stein and Cohen (1988) Oligodeoxynucleotides as inhibitors of gene expression: a review Cancer Res. 48:2659-2668
Takabe et al. (2003) Adenovirus-mediated overexpression of follistatin enlarges intact liver of adult rats. Hepatology 38:1107-1115
van der Krol et al. (1988) Modulation of eukaryotic gene expression by complementary RNA or DNA sequences. Biotechniques 6:958-976
Xu et al. (1995) Inhibin antagonizes inhibition of liver cell growth by activin in a dominant-negative mechanism. J. Biol. Chem. 270:6308-6313

1. A method of reducing airway tissue mucus secretion in a mammal, said method comprising downregulating the functional level of activin or upregulating the functional level of follistatin in said mammal.

2. A method of therapeutically or prophylactically treating a condition which is characterised by airway tissue mucus dysfunction, said method comprising downregulating the functional level of activin or upregulating the functional level of follistatin in said mammal wherein downregulating said level of activin or upregulating said follistatin reduces airway tissue mucus secretion.

3. (canceled)

4. The method according to claim 1, wherein said activin antagonist or follistatin levels are the levels in the airway tissue of said mammal.

5. The method according to claim 1, wherein said airway tissue is lung tissue.

6. The method according to claim 1, wherein said mucus secretion is mucus hypersecretion.

7. The method according to claim 1, wherein said activin is activin A or activin B.

8. The method according to claim 1, wherein the functional level of activin is downregulated by an activin antagonist selected from inhibin, the activin βc subunit, the α subunit of inhibin, an antibody directed to activin, a non-functional activin mutant, a non-functional activin receptor mutant, and a soluble activin receptor.

9. (canceled)

10. The method according to claim 1, wherein the functional level of activin is downregulated by a proteinaceous or non-proteinaceous molecule which downregulates the transcription or translation of the activin gene.

11. The method according to claim 10, wherein:
(a) said proteinaceous molecule is an antibody directed to activin DNA or mRNA; or
(b) said non-proteinaceous molecule is an activin antisense oligonucleotide, a DNAzyme, an aptamer, or a molecule that cosuppresses activin expression.

12. (canceled)

13. The method according to claim 1, wherein the functional level of follistatin is upregulated by:
(a) follistatin or functional fragment thereof; or
(b) increasing the transcription or translation of follistatin.

14. The method according to claim 13, wherein said follistatin is FS315 or FS288.

15. (canceled)

16. The method according to claim 13, wherein the follistatin is expressed in vivo by an exogenous genetic construct.

17. The method according to claim 2, wherein said condition is a non-inflammatory condition.

18. The method according to claim 2, wherein:
(a) said mucus secretion occurs prior to the onset of inflammation or is regulated by non-inflammatory mechanisms; and/or
(b) the mucus secretion levels are reduced are normal levels.

19. (canceled)

20. The method according to claim 2 wherein said condition is:
(a) one in which lung clearance mechanisms are disrupted; or
(b) selected from asthma, cystic fibrosis, chronic obstructive pulmonary disease, bronchiectasis, primary ciliary dyskinesia, panbtonchiolitis, pulmonary hypertension, idiopathic pulmonary fibrosis immunodeficiency states, hypogammaglobulinemia, human immunodeficiency virus infection, organ transplantation, hematologic malignant conditions, intubation, impaired mucus clearance, disruption of lung clearance mechanisms as a result of paralysis, immobilization and surgery.

21. (canceled)

22. The method according to claim 8, wherein said activin antagonist is administered systemically.

23. The method according to claim 8 wherein said activin antagonist administration is localised to the airway tissue.

24. (canceled)

25. The method according to claim 23 wherein said administration is through the nose or mouth.

26. The method according to claim 25 wherein said administration is by inhalation of an aerosol or is by a liquid delivery system or nebulizer.

27. The method according to claim 1, wherein said mammal is a human.
